(19) United States
(12) Patent Application Publication
(10) Pub. No.: US 2020/0330615 A1
(43) Pub. Date: Oct. 22, 2020

(54) NONINVASIVE IMAGING OF FOCAL ATHEROSCLEROTIC LESIONS USING FLUORESCENCE MOLECULAR TOMOGRAPHY

(71) Applicant: Washington University, St. Louis, MO (US)

(72) Inventors: Pamela K. Woodard, St. Louis, MO (US); Monica Shokeen, St. Louis, MO (US); Samuel Achilefu, St. Louis, MO (US)

(21) Appl. No.: 16/919,540

(22) Filed: Jul. 2, 2020

Related U.S. Application Data
(63) Continuation of application No. 15/287,316, filed on Oct. 6, 2016.
(60) Provisional application No. 62/237,937, filed on Oct. 6, 2015.

(51) Int. Cl. A61K 49/00

(52) U.S. Cl. CPC A61K 49/0056; A61K 49/0032

(57) ABSTRACT

The present disclosure provides compositions comprising an oligopeptide comprising a fragment of a natriuretic peptide, wherein the fragment comprises the sequence Arg-Ile-Asp-Arg-Ile (SEQ ID NO:1), and a detectable label. Further disclosed are methods of imaging atherosclerotic plaque by optical imaging using a peptide composition.

### CROSS REFERENCE TO RELATED APPLICATIONS

This application is a continuation of U.S. patent application Ser. No. 15/287,316, filed Oct. 6, 2016, which claims the benefit of U.S. Provisional Application No. 62/237,937, filed Oct. 6, 2015, the disclosures of which are hereby incorporated by reference in their entirety.

### GOVERNMENTAL RIGHTS

This invention was made with government support under HHSN268201000046C awarded by the NIH. The government has certain rights in the invention.

### FIELD OF THE INVENTION

The present disclosure provides compositions comprising an oligopeptide comprising a fragment of a natriuretic peptide, wherein the fragment comprises the sequence Arg-Ile-Asp-Arg-Ile (SEQ ID NO:1), and a detectable label. Further disclosed are methods of imaging atherosclerotic plaque by optical imaging using a peptide composition.

### BACKGROUND OF THE INVENTION

Cardiovascular disease is the leading cause of death worldwide despite primary and second prevention. Each year, more than 1 million people in the United States and over 19 million people worldwide experience a sudden cardiac event (acute coronary syndromes and/or sudden cardiac death). A large portion of these individuals have had no prior symptoms.

Atherosclerosis is a systemic disease characterized by accumulation of lipids, inflammatory cells, and connective tissue within the arterial wall. It is a chronic, progressive disease with a long asymptomatic phase. Atherosclerotic plaque exists in at least two forms: unstable (“soft”) plaque, and calcified plaque. Unstable plaque is characterized by an eccentric neo-intimal lesion with a lipid core covered by a thinning cap of smooth muscle cells, active angiogenesis, increased matrix metalloproteinase activity, and translocation of monocyte/macrophages that transform into foam cells.

Soft plaque can rupture and such ruptures can lead to rapid death. Rupture of an atherosclerotic plaque accounts for approximately 70% of the severe clinical events such as stroke or fatal acute myocardial infarction and/or sudden coronary death. Timely noninvasive imaging that could signal prerupture plaque progression will reduce the morbidity and mortality by allowing early intervention. Although positron emission tomography (PET) and magnetic resonance imaging (MRI) are routinely used for metabolic and morphologic imaging, these modalities are not suited for frequent monitoring or even screening of at-risk patients because of ionizing radiation (PET) and expense (PET, MRI). Transcutaneous Doppler and intravascular ultrasound are insensitive to the subtle molecular changes of critical importance. Thus there is a need in the art for modalities to image atherosclerotic plaques that are noninvasive and are useful to detect molecular changes in the plaque suggestive of plaque instability pre-rupture.

### SUMMARY OF THE INVENTION

In an aspect, the disclosure provides a composition comprising a peptide conjugated to a detectable label, wherein the peptide is capable of binding C-type natriuretic peptide receptors (NPR-C) and comprises the sequence Arg-Ile-Asp-Arg-Ile (SEQ ID NO:1) and wherein the detectable label is a NIR dye.

In another aspect, the disclosure provides a method to determine distribution of C-type natriuretic peptide receptors (NPR-C) in a subject, the method comprising: administering to the subject a peptide conjugated to a detectable label, wherein the peptide is capable of binding C-type natriuretic peptide receptors (NPR-C) and comprises the sequence Arg-Ile-Asp-Arg-Ile (SEQ ID NO:1) and wherein the detectable label is a NIR dye; and imaging the subject to detect the presence of a signal emitted from the peptide, wherein the signal being emitted is from binding of the peptide to one or more C-type natriuretic peptide receptors.

In still another aspect, the disclosure provides a method to treat a pathological condition associated with expression of C-type natriuretic peptide receptors (NPR-C) in a subject, the method comprising administering to the subject a peptide conjugated to a therapeutic agent, wherein the peptide is capable of binding C-type natriuretic peptide receptors (NPR-C) and comprises the sequence Arg-Ile-Asp-Arg-Ile (SEQ ID NO:1).

### BRIEF DESCRIPTION OF THE FIGURES

The application file contains at least one drawing executed in color. Copies of this patent application publication with color drawing(s) will be provided by the Office upon request and payment of the necessary fee.

FIG. 1A depicts a schematic of the construct used for imaging: Cypate-Arg-Ser-Ser-c[Cys-Phe-Gly-Gly-Arg-Ile-Asp-Arg-Ile-Gly-Ala-Cys]-NH2 (SEQ ID NO:3). FIG. 1B depicts a graph showing the absorption and fluorescence spectra of LS668 in dimethylsulfoxide. FIG. 1C, FIG. 1D and FIG. 1E depict fluorescence microscopy images showing cellular internalization of LS668 (FIG. 1C) in NPR-C transfected cells, (FIG. 1D) inhibition of internalization in presence of excess C-ANF peptide, and (FIG. 1E) absence of internalization in NPR-A transfected cells. Blue (DAPI, nuclear stain) and red (LS668). Scale: 100 m.

FIG. 2A, FIG. 2B, FIG. 2C, FIG. 2D, FIG. 2E, FIG. 2F, FIG. 2G, FIG. 2H and FIG. 2I depict images showing coronal (depth=7 mm), sagittal and transverse sections of reconstructed fluorescence molecular tomography (FMT) signal from injured artery (FIG. 2A, FIG. 2B, FIG. 2C) and corresponding control artery (FIG. 2D, FIG. 2E, FIG. 2F) from a representative animal (rabbit 1). White lines indicate the position of the respective sagittal and transverse sections. FIG. 2G depicts a schematic showing the relationship between the FMT images displayed to their orientation with respect to the tissue volume. FIG. 2H depicts a graph showing time dependent changes in integrated fluorescence signal (mean SD, n=3) for injured and control arteries (*P=0.0283; **P=0.0282). FIG. 2I depicts a graph showing the mean (n=2) fluorescence intensity obtained from the ex vivo injured artery containing the lesion and the control artery. Adjoining figure (inset) shows the fluorescence images (excitation/emission: 785 nm/>800 nm) of the injured artery containing the lesion (top) and the control artery (bottom).

FIG. 3A, FIG. 3B and FIG. 3C depict images showing ex vivo studies on the paraffin fixed sections of injured (top row) and control artery (bottom row) sections obtained at 8 weeks postsurgery. FIG. 3A depicts bright field images showing IEL, internal elastic lamina; A, adventitia; M, media; 1° NEO, primary neointima. Scale: 500 m. FIG. 3B depicts corresponding fluorescence images (excitation/emission: 710 75 nm 810 90 nm) after ex vivo staining with LS668. Scale: 500 m. FIG. 3C depicts immunohistochemistry on tissue sections with clone RAM11 antibody (1:100 dilution; blue) for macrophages and counterstained with nuclear fast red. Scale: 250 m.

### DETAILED DESCRIPTION OF THE INVENTION

The inventors herein have succeeded in devising new peptide compositions which can be used for imaging distribution of natriuretic peptide receptors, including receptors which bind C-type atrial natriuretic factor (CANF). In some embodiments, these peptide compositions can be used for imaging and monitoring angiogenesis during the course of anti-angiogenic treatment of cancer. In other embodiments, these peptide compositions can be used for imaging and monitoring the presence and progression of atherosclerosis, including imaging of atherosclerotic plaque. In various embodiments, the peptide compositions described herein can be used as probes for imaging angiogenesis or atherosclerosis using optical imaging.

### I. Composition

In an aspect, the disclosure provides a composition comprising a peptide conjugated to a detectable label and/or therapeutic agent. Specifically, the peptide is capable of binding C-type natriuretic peptide receptors (NPR-C) and comprises the sequence Arg-Ile-Asp-Arg-Ile (SEQ ID NO:1). The peptide may be cyclic or linear and the peptide may be conjugated to the detectable label and/or therapeutic agent directly via a covalent bond or indirectly via a linker. Various aspects of the composition are described in more detail below.

(a) Peptide

In an aspect, the present disclosure provides a peptide conjugated to a detectable label, wherein the peptide is capable of binding natriuretic peptide receptors. More specifically, the peptide is capable of binding the C-type natriuretic peptide receptors (NPR-C). In certain embodiments, the peptide is a C-type atrial natriuretic peptide or a fragment thereof. The term “C-type atrial natriuretic peptide” may be used interchangeably with C-type atrial natriuretic factor, CANP, or CANF. By “peptide” is meant an amino acid sequence that includes 5 or more amino acid residues. “Peptide” refers to both short chains, commonly referred to as peptides, oligopeptides, or oligomers, and to longer chains, up to about 100 residues in length. In some aspects, the peptide can have the sequence of a C-type atrial natriuretic peptide or a fragment thereof. In various configurations, such peptides can comprise the sequence Arg-Ile-Asp-Arg-Ile (SEQ ID NO:1). Thus, in some embodiments, the peptide can be a fragment that is less than a full-length natriuretic peptide. In an embodiment, the peptide is linear. In another embodiment, the peptide is cyclic.

In various embodiments, the peptide can include at least 2 cysteine residues, which can comprise, in various configurations, at least one cystine (i.e., including a disulfide bridge). In some other configurations, the cysteines can be in reduced form (i.e., not including a disulfide bridge). In some configurations, a peptide can comprise the sequence Cys-Phe-Gly-Gly-Arg-Ile-Asp-Arg-Ile-Gly-Ala-Cys-NH2 (SEQ ID NO:2), in which the carboxy terminal cysteine is aminated. In some configurations, the cysteines of this sequence can comprise a disulfide linkage (a cystine). In some configurations, the cysteines of these peptides can comprise a cysteine comprising a disulfide bridge, or can be in the reduced, free sulthydryl form. In some configurations, the peptide moiety can be a fragment of a natriuretic peptide and consist of the sequence H-Arg-Ser-Ser-c[Cys-Phe-Gly-Gly-Arg-Ile-Asp-Arg-Ile-Gly-Ala-Cys]-NH2 (SEQ ID NO:3). In addition, a peptide can further comprise a sequence unrelated to natriuretic peptide.

In various configurations, a peptide can be no greater than 25 amino acids, no greater than 24 amino acids, no greater than 23 amino, acids, no greater than 22 amino acids, no greater than 21 amino acids, no greater than 20 amino acids, no greater than 19 amino acids, no greater than 18 amino acids, no greater than 17 amino acids, no greater than 16 amino acids, no greater than 15 amino acids, no greater than 14 amino acids, no greater than 13 amino acids, no greater than 12 amino acids, no greater than 11 amino acids, or no greater than 10 amino acids in length. In another embodiment, a peptide is 25, 24, 23, 22, 21, 20, 19, 18, 17, 16, 15, 14, 13, 12, 11, 10, 9, 8, 7, 6 or 5 amino acids. In other embodiments, a peptide is about 25 to about 20 amino acids, about 25 to about 15 amino acids, about 25 to about 10 amino acids, about 25 to about 5 amino acids, about 20 to about 15 amino acids, about 20 to about 10 amino acids, about 20 to about 5 amino acids, about 15 to about 10 amino acids, about 15 to about 5 amino acids, or about 10 to about 5 amino acids. In other embodiments, a peptide is 25 to 20 amino acids, 25 to 15 amino acids, 25 to 10 amino acids, 25 to 5 amino acids, 20 to 15 amino acids, 20 to 10 amino acids, 20 to 5 amino acids, 15 to 10 amino acids, 15 to 5 amino acids, or 10 to 5 amino acids.

A peptide of the disclosure may be subject to various changes, substitutions, insertions, and deletions where such changes provide for certain advantages in its use. Thus, the disclosure encompasses any of a variety of forms of peptide derivatives that include amides, conjugates with proteins, cyclized peptides, polymerized peptides, conservatively substituted variants, analogs, fragments, peptoids, chemically modified peptides, peptide mimetics, and replacement of Adenoviral knob (See, for example, Mathis et al., Oncogene 2005; 24:7775-7791).

Peptides of the disclosure may comprise naturally occurring amino acids, synthetic amino acids, genetically encoded amino acids, non-genetically encoded amino acids, and combinations thereof. Peptides may include both L-form and D-form amino acids.

Representative non-genetically encoded amino acids may include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids may include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

The term “conservatively substituted variant” refers to a peptide comprising an amino acid residue sequence similar to a sequence of a reference peptide that binds a natriuretic peptide receptor in which one or more residues have been conservatively substituted with a functionally similar residue and which displays the targeting activity as described herein. The phrase “conservatively substituted variant” also includes peptides wherein a residue is replaced with a chemically derivatized residue, provided that the resulting peptide displays targeting activity as disclosed herein.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Peptides of the present disclosure also include peptides comprising one or more additions and/or deletions or residues relative to the sequence of a peptide whose sequence is disclosed herein, so long as the requisite targeting activity of the peptide is maintained. The term “fragment” refers to a peptide comprising an amino acid residue sequence shorter than that of a peptide disclosed herein.

The term “peptoid” as used herein refers to a peptide wherein one or more of the peptide bonds are replaced by pseudopeptide bonds including but not limited to a carba bond (CH₂—CH₂), a depsi bond (CO—O), a hydroxyethylene bond (CHOH—CH₂), a ketomethylene bond (CO—CH₂), a methylene-oxy bond (CH₂—O), a reduced bond (CH₂—NH), a thiomethylene bond (CH₂—S), a thiopeptide bond (CS—NH), and an N-modified bond (—NRCO—). See e.g. Corringer et al. (1993) J Med Chem 36:166-172; Garbay-Jauregiuberry et al. (1992) Int J Pept Protein Res 39:523-527; Tung et al. (1992) Pept Res 5:115-118; Urge et al. (1992) Carbohydr Res 235:83-93; Pavone et al. (1993) Int J Pept Protein Res 41:15-20.

Peptides of the present disclosure, including peptoids, may be synthesized by any of the techniques that are known to those skilled in the art of peptide synthesis. Synthetic chemistry techniques, such as a solid-phase Merrifield-type synthesis, may be preferred for reasons of purity, antigenic specificity, freedom from undesired side products, ease of production and the like. A summary of representative techniques can be found in Stewart & Young (1969) Solid Phase Peptide Synthesis. Freeman, San Francisco; Merrifield (1969) Adv Enzymol Relat Areas Mol Biol 32:221-296; Fields & Noble (1990) Int J Pept Protein Res 35:161-214; and Bodanszky (1993) Principles of Peptide Synthesis. 2nd rev. ed. Springer-Verlag, Berlin; New York. Solid phase synthesis techniques can be found in Andersson et al. (2000) Biopolymers 55:227-250, references cited therein, and in U.S. Pat. Nos. 6,015,561, 6,015,881, 6,031,071, and 4,244,946. Peptide synthesis in solution is described by SchrOder & LUbke (1965) The Peptides. Academic Press, New York. Appropriate protective groups usable in such synthesis are described in the above texts and in McOmie (1973) Protective Groups in Organic Chemistry. Plenum Press, London, New York. Peptides that include naturally occurring amino acids can also be produced using recombinant DNA technology. In addition, peptides comprising a specified amino acid sequence can be purchased from commercial sources (e.g., Biopeptide Co., LLC of San Diego, Calif. and PeptidoGenics of Livermore, Calif.).

Any peptide or peptide mimetic of the present disclosure may be used in the form of a pharmaceutically acceptable salt. Suitable acids which are capable of forming a pharmaceutically acceptable salt with the peptides of the present invention include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Suitable bases capable of forming salts with the peptides of the present disclosure include inorganic bases such as sodium hydroxide, ammonium hydroxide, potassium hydroxide and the like; and organic bases such as mono-di- and tri-alkyl and aryl amines (e.g. triethylamine, diisopropyl amine, methyl amine, dimethyl amine and the like), and optionally substituted ethanolamines (e.g. ethanolamine, diethanolamine and the like).

(b) Detectable Label

A peptide of the disclosure is conjugated to a detectable label. The detectable label may be directly conjugated to the peptide or may be indirectly conjugated to the peptide. In an embodiment, the detectable label may be complexed with a chelating agent that is conjugated to the peptide. In another embodiment, the detectable label may be complexed with a chelating agent that is conjugated to a linker that is conjugated to the peptide. In still another embodiment, the detectable label may be conjugated to a linker that is conjugated to the peptide. In certain embodiments, more than one peptide is conjugated to the detectable label via more than one linker. For example, 1, 2, 3, 4, or 5 peptides may be conjugated to the detectable label via 1, 2, 3, 4, or 5 linkers. In certain embodiments, 2 peptides are conjugated to the detectable label via 2 linkers. In still yet another embodiment, a detectable label may be indirectly attached to a peptide of the disclosure by the ability of the label to be specifically bound by a second molecule. One example of this type of an indirectly attached label is a biotin label that can be specifically bound by the second molecule, streptavidin. Single, dual or multiple labeling may be advantageous.

As used herein, a “detectable label” is any type of label which, when attached to a peptide of the renders the peptide detectable. In general, detectable labels may include luminescent molecules, chemiluminescent molecules, fluorochromes, fluorophores, fluorescent quenching agents, colored molecules, radioisotopes, radionuclides, cintillants, massive labels such as a metal atom (for detection via mass changes), biotin, avidin, streptavidin, protein A, protein G, antibodies or fragments thereof, Grb2, polyhistidine, Ni²⁺, Flag tags, myc tags, heavy metals, enzymes, alkaline phosphatase, peroxidase, luciferase, electron donors/acceptors, acridinium esters, and colorimetric substrates. In a specific embodiment, the detectable label is an optical imaging agent. Non-limiting examples of optical imaging agents include fluorophores, organic fluorescent dyes, luminescent imaging agents, fluorescent lanthanide complexes, and fluorescent semiconductor nanocrystals. The skilled artisan would readily recognize other useful labels that are not mentioned above, which may be employed in the operation of the present invention.

A detectable label emits a signal that can be detected by a signal transducing machine. In some cases, the detectable label can emit a signal spontaneously, such as when the detectable label is a radionuclide. In other cases the detectable label emits a signal as a result of being stimulated by an external field such as when the detectable label is a relaxivity metal. Examples of signals include, without limitation, gamma rays, X-rays, visible light, infrared energy, and radiowaves. Examples of signal transducing machines include, without limitation, gamma cameras including SPECT/CT devices, PET scanners, fluorimeters, and Magnetic Resonance Imaging (MRI) machines. As such, the detectable label comprises a label that can be detected using magnetic resonance imaging, scintigraphic imaging, ultrasound, fluorescence, or fluorescence molecular tomography (FMT). FMT is an optical imaging technology that allows tomographic and quantitative visualization of molecular events in vivo.

Suitable fluorophores include, but are not limited to, fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), DyDelight Dyes, near infrared (NIR) (700-900 nm) fluorescent dyes, and carbocyanine and aminostyryl dyes. In a specific embodiment, the detectable label is a NIR fluorescent dye cypate. For example, see U.S. Pat. Nos. 8,344,158; 8,318,133; and US 2009/0214436, each of the disclosures of which are hereby incorporated by reference in their entirety. A peptide of the disclosure can be labeled for fluorescence detection by labeling the agent with a fluorophore using techniques well known in the art (see, e.g., Lohse et al., Bioconj Chem 8:503-509 (1997)). For example, many known dyes are capable of being coupled to NH2-terminal amino acid residues. Alternatively, a fluorochrome such as fluorescein may be bound to a lysine residue of the peptide linker.

A radionuclide may be a γ-emitting radionuclide, Auger-emitting radionuclide, β-emitting radionuclide, an α-emitting radionuclide, or a positron-emitting radionuclide. A radionuclide may be a detectable label and/or a cytotoxic agent. Non-limiting examples of suitable radionuclides may include carbon-11, nitrogen-13, oxygen-15, fluorine-18, fluorodeoxyglucose-18, phosphorous-32, scandium-47, copper-64, 65 and 67, gallium-67 and 68, bromine-75, 77 and 80m, rubidium-82, strontium-89, zirconium-89, yttrium-86 and 90, ruthenium-95, 97, 103 and 105, rhenium-99m, 101, 105, 186 and 188, technetium-99m, rhodium-105, mercury-107, palladium-109, indium-111, silver-111, indium-113m, lanthanide-114m, tin-117m, tellurium-121m, 122m and 125m, iodine-122, 123, 124, 125, 126, 131 and 133, praseodymium-142, promethium-149, samarium-153, gadolinium-159, thulium-165, 167 and 168, dysprosium-165, holmium-166, lutetium-177, rhenium-186 and 188, iridium-192, platinum-193 and 195m, gold-199, thallium-201, titanium-201, astatine-211, bismuth-212 and 213, lead-212, radium-223, actinium-225, and nitride or oxide forms derived there from. In a specific embodiment, a radionuclide is selected from the group consisting of copper-64, zirconium-89, yttrium-90, indium-111, and lutetium-177. In another specific embodiment, a radionuclide is selected from the group consisting of yttrium-90, indium-111, and lutetium-177.

A variety of metal atoms may be used as a detectable label. The metal atom may generally be selected from the group of metal atoms comprised of metals with an atomic number of twenty or greater. For instance, the metal atoms may be calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms. In some embodiments, the metal atoms may be selected from the group comprising alkali metals with an atomic number greater than twenty. In other embodiments, the metal atoms may be selected from the group comprising alkaline earth metals with an atomic number greater than twenty. In one embodiment, the metal atoms may be selected from the group of metals comprising the lanthanides. In another embodiment, the metal atoms may be selected from the group of metals comprising the actinides. In still another embodiment, the metal atoms may be selected from the group of metals comprising the transition metals. In yet another embodiment, the metal atoms may be selected from the group of metals comprising the poor metals. In other embodiments, the metal atoms may be selected from the group comprising gold atoms, bismuth atoms, tantalum atoms, and gadolinium atoms. In preferred embodiments, the metal atoms may be selected from the group comprising metals with an atomic number of 53 (i.e. iodine) to 83 (i.e. bismuth). In an alternative embodiment, the metal atoms may be atoms suitable for magnetic resonance imaging. In another alternative embodiment, the metal atoms may be selected from the group consisting of metals that have a K-edge in the x-ray energy band of CT. Preferred metal atoms include, but are not limited to, manganese, iron, gadolinium, gold, and iodine.

The metal atoms may be metal ions in the form of +1, +2, or +3 oxidation states. For instance, non-limiting examples include Ba²⁺, Bi³⁺, Cs+, Ca²⁺, Cr²⁺, Cr³⁺, Cr⁶⁺, Co²⁺, Co³⁺, Cu+, Cu²⁺, Cu³⁺, Ga³⁺, Gd³⁺, Au+, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag+, Sr²⁺, Sn²⁺, Sn⁴⁺, and Zn²⁺. The metal atoms may comprise a metal oxide. For instance, non-limiting examples of metal oxides may include iron oxide, manganese oxide, or gadolinium oxide. Additional examples may include magnetite, maghemite, or a combination thereof.

A peptide comprising a chelating agent may incorporate a radionuclide or metal atom. Incorporation of the radionuclide or metal atom with a peptide-chelating agent complex may be achieved by various methods common in the art of coordination chemistry. For example, when the metal is technetium-99m, the following general procedure may be used to form a technetium complex. A peptide-chelating agent complex solution is formed initially by dissolving the complex in aqueous alcohol such as ethanol. The solution is then degassed to remove oxygen then thiol protecting groups are removed with a suitable reagent, for example, with sodium hydroxide, and then neutralized with an organic acid, such as acetic acid (pH 6.0-6.5). In the labeling step, a stoichiometric excess of sodium pertechnetate, obtained from a molybdenum generator, is added to a solution of the complex with an amount of a reducing agent such as stannous chloride sufficient to reduce technetium and heated. The labeled complex may be separated from contaminants ^{99m}TcO₄⁻ and colloidal ^{99m}TcO₂ chromatographically, for example, with a C-18 Sep Pak cartridge.

In an alternative method, labeling can be accomplished by a transchelation reaction. The technetium source is a solution of technetium complexed with labile ligands facilitating ligand exchange with the selected chelator. Suitable ligands for transchelation include tartarate, citrate, and heptagluconate. In this instance the preferred reducing reagent is sodium dithionite. It will be appreciated that the complex may be labeled using the techniques described above, or alternatively the chelator itself may be labeled and subsequently conjugated to the peptide to form the complex; a process referred to as the “prelabeled ligand” method.

Another approach for labeling complexes of the present disclosure involves immobilizing the peptide-chelating agent complex on a solid-phase support through a linkage that is cleaved upon metal chelation. This is achieved when the chelating agent is coupled to a functional group of the support by one of the complexing atoms. Preferably, a complexing sulfur atom is coupled to the support which is functionalized with a sulfur protecting group such as maleimide.

Still another approach for labeling peptides involves incubation with a desired radionuclide. For example, a peptide comprising a linker, one or more chelators and PEG may be dissolved in ammonium acetate buffer. Ammonium acetate may be added to a ¹¹¹InCl₃ stock solution and carefully mixed; the final pH should be between about 5.5-5.8. The ¹¹¹InCl₃ may then be added to the peptide at a ratio of about 370:1 kBq:μg and the reaction mixture may be incubated at about 95° C. with constant shaking for about 1 h. The radiolabeling efficiency of the peptide construct may be determined using instant thin-layer chromatography.

In another embodiment, a detectable label may be conjugated directly or indirectly to a peptide without the use of a chelating agent. For example, the detectable label is conjugated to a linker that is conjugated to a peptide. For example, a radioactive iodine label (e.g., ¹²²I, ¹²³I, ¹²⁴I, ¹²⁵I, or ¹³¹I)) is capable of being conjugated to each D- or L-Tyr or D- or L-4-amino-Phe residue present in a peptide linker of the disclosure. In an embodiment, a tyrosine residue of a peptide linker of the disclosure may be halogenated. Halogens include fluorine, chlorine, bromine, iodine, and astatine. Such halogenated peptides of the disclosure may be detectably labeled if the halogen is a radioisotope, such as, for example, ¹⁸F, ⁷⁵Br, ⁷⁷Br, ¹²²I, ¹²³I, ¹²⁴I, ¹²⁵I, ¹²⁹I, ¹³¹I, or ²¹¹At. Halogenated peptides of the disclosure contain a halogen covalently bound to at least one amino acid, and preferably to D-Tyr residues present in the peptide linker.

(c) Therapeutic Agent

A peptide of the disclosure optionally includes one or more therapeutic agents. Accordingly, the peptide may be conjugated to a detectable label, a therapeutic agent, or a detectable label and therapeutic agent. The therapeutic agent may be directly conjugated to the peptide or may be indirectly conjugated to the peptide. In certain embodiments, the therapeutic agent may be conjugated to a linker that is conjugated to the peptide. In an embodiment, more than one therapeutic agent is conjugated to the peptide via more than one linker.

As will be appreciated by the skilled artisan, the choice of a particular therapeutic agent can and will vary depending upon the indication to be treated and its stage of progression. Because the peptides of the disclosure are selectively targeted to natriuretic peptide receptors, the therapeutic agents are generally directed toward treatment of inflammation and cardiovascular disease. For example, when the indication is inflammation, the therapeutic agent may be an NSAID such as aniline derivatives (acetomenaphin), indole-3-acetic acid derivatives (indomethacin), specific Cox-2 inhibitors (Celebrex), and aspirin. Alternatively, when the indication is cardiovascular disease, the therapeutic agent may include sodium-channel blockers (e.g., quinidine), beta-blockers (e.g., propranolol), calcium-channel blockers (e.g., diltiazen), diuretics (e.g., hydrochlorothiazide), ACE inhibitors (e.g., captopril), and thrombolytic agents (e.g., tissue plasminogen activator and streptokinase).

(d) Linker

C-terminus of cypate is conjugated to N-terminus of PEG4 (Fmoc-PEG-COOH). Then the C-terminus of PEG4 is conjugated to the N-terminus of CANF peptide.

A peptide of the disclosure may be conjugated to a detectable label and/or therapeutic agent via a linker. It is to be understood that conjugation of the peptide to the linker and conjugation of the linker to the detectable label and/or therapeutic agent will not adversely affect either the targeting function of the peptide or the activity of the detectable label and/or therapeutic agent. In certain embodiments, the peptide is conjugated to the linker via its N-terminus and the detectable label is conjugated to the linker via its C-terminus. Specifically, the C-terminus of the detectable label is conjugated to the N-terminus of the linker and then the C-terminus of the linker is conjugated to the N-terminus of the peptide. Suitable linkers include amino acid chains and alkyl chains functionalized with reactive groups for coupling to both the peptide and the detectable label and/or therapeutic agent.

In an embodiment, the linker may include amino acid side chains, referred to as a peptide linker. Accordingly, additional amino acid residues may be added at the amino terminus of a peptide of the disclosure for the purpose of providing a linker by which the peptides of the present disclosure can be conveniently affixed to a detectable label or therapeutic agent. Importantly, an amino acid linker alone does not specifically bind to natriuretic peptide receptors. Amino acid residue linkers are usually at least one residue and can be 50 or more residues, but alone do not specifically bind to the target protein. In an embodiment, a linker may be about 1 to about 10 amino acids. In another embodiment, a linker may be about 10 to about 20 amino acids. In still another embodiment, a linker may be about 20 to about 30 amino acids. In still yet another embodiment, a linker may be about 30 to about 40 amino acids. In different embodiments, a linker may be about 40 to about 50 amino acids. In other embodiments, a linker may be more than 50 amino acids. For instance, a linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. In a specific embodiment, a linker is about 20 to about 30 amino acids. In another specific embodiment, a linker is about 26 amino acids.

Any amino acid residue may be used for the linker provided the linker does not specifically bind to natriuretic peptide receptors. Typical amino acid residues used for linking are glycine, serine, alanine, leucine, tyrosine, cysteine, lysine, glutamic and aspartic acid, or the like. For example, a linker may be (AAS)ₙ, (AAAL)ₙ, (GₙS)ₙ or (G₂S)ₙ, wherein A is alanine, S is serine, L is leucine, and G is glycine and wherein n is an integer from 1-20, or 1-10, or 3-10. Accordingly, n may be 1, 2, 3, 4, 5, 6, 7, 8, 9, 10, 11, 12, 13, 14, 15, 16, 17, 18, 19 or 20. Thus, in certain embodiments, a linker includes, but is not limited to, (AAS)ₙ, (AAAL)ₙ, (GnS)ₙ or (G₂S)ₙ, wherein A is alanine, S is serine, L is leucine, and G is glycine and wherein n is an integer from 1-20, or 1-10, or 3-10.

In another embodiment, an alkyl chain linking group may be conjugated to the peptide by reacting the terminal amino group or the terminal carboxyl group with a functional group on the alkyl chain, such as a carboxyl group or an activated ester. Subsequently the detectable label and/or therapeutic agent is attached to the alkyl chain to complete the formation of the complex by reacting a second functional group on the alkyl chain with an appropriate group on the detectable label and/or therapeutic agent. The second functional group on the alkyl chain is selected from substituents that are reactive with a functional group on the detectable label and/or therapeutic agent while not being reactive with the peptide. For example, when the detectable label and/or therapeutic agent incorporates a functional group, such as a carboxyl group or an activated ester, the second functional group of the alkyl chain linking group can be an amino group or vice versa. It will be appreciated that formation of the conjugate may require protection and deprotection of the functional groups present in order to avoid formation of undesired products. Protection and deprotection are accomplished using protecting groups, reagents, and protocols common in the art of organic synthesis. Particularly, protection and deprotection techniques employed in solid phase peptide synthesis may be used. It will be appreciated that linking groups may alternatively be coupled first to the detectable label and/or therapeutic agent and then to the peptide.

An alternative chemical linking group to an alkyl chain is polyethylene glycol (PEG), which is functionalized in the same manner as the alkyl chain described above. Such a linker may be referred to as a heterobifunctional PEG linker or a homobifunctional PEG linker. Non-limiting examples of heterobifunctional PEG linkers include: O-(2-Aminoethyl)-O′-[2-(biotinylamino)ethyl]octaethylene glycol; O-(2-Aminoethyl)-O′-(2-carboxyethyl)polyethylene glycol hydrochloride Mₚ 3000; O-(2-Aminoethyl)-O′-(2-carboxyethyl)polyethylene glycol 5,000 hydrochloride Mₚ 5,000; O-(2-Aminoethyl)polyethylene glycol 3,000 Mp 3,000; O-(2-Aminoethyl)-O′-(2-(succinylamino)ethyl)polyethylene glycol hydrochloride Mₚ 10,000; O-(2-Azidoethyl)heptaethylene glycol; O-[2-(Biotinylamino)ethyl]-O′-(2-carboxyethyl)undecaethylene glycol; 21-[D(+)-Biotinylamino]-4,7,10,13,16,19-hexaoxaheneicosanoic acid; O-(2-Carboxyethyl)-O′-[2-(Fmoc-amino)-ethyl]heptacosaethylene glycol; O-(2-Carboxyethyl)-O′-(2-mercaptoethyl)heptaethylene glycol; O-(3-Carboxypropyl)-O′-[2-(3-mercaptopropionylamino)ethyl]-polyethylene glycol Mw 3000; O-(3-Carboxypropyl)-O′-[2-(3-mercaptopropionylamino)ethyl]-polyethylene glycol Mw 5000; O—[N-(3-Maleimidopropionyl)aminoethyl]-O′-[3-(N-succinimidyloxy)-3-oxopropyl]heptacosaethylene glycol; and O-[2-(3-Tritylthiopropionylamino)ethyl]polyethylene glycol Mₚ 3,000. Non-limiting examples of homobifunctional PEG linkers include: MAL-PEG-MAL (Bifunctional Maleimide PEG Maleimide); OPSS-PEG-OPSS (OPSS: orthopyridyl disulfide; PDP-PEG-PDP); HS-PEG-SH (Bifunctional Thiol PEG Thiol); SG-PEG-SG (Bifunctional PEG Succinimidyl Glutarate NHS ester); SS-PEG-SS (Bifunctional PEG Succinimidyl Succinate NHS ester); GAS-PEG-GAS (Bifunctional PEG Succinimidyl ester NHS-PEG-NHS); SAS-PEG-SAS (Bifunctional PEG Succinimidyl ester NHS-PEG-NHS); Amine-PEG-Amine (Bifunctional PEG Amine NH2-PEG-NH2); AC-PEG-AC (Bifunctional Acrylate PEG Acrylate); ACA-PEG-ACA (Bifunctional Polymerizable PEG Acrylate Acrylamide); Epoxide-PEG-Epoxide (Bifunctional PEG Epoxide or EP); NPC-PEG-NPC (Bifunctional NPC PEG, Nitrophenyl Carbonate); Aldehyde-PEG-Aldehyde (ALD-PEG-ALD, bifunctional PEG propionaldehyde); AA-PEG-AA (Acid-PEG-Acid, AA-acetic acid or carboxyl methyl); GA-PEG-GA (Acid-PEG-Acid, GA: Glutaric acid); SA-PEG-SA (Bifunctional PEG carboxylic acid-Succinic Acid); GAA-PEG-GAA (Bifunctional PEG carboxylic acid, Glutaramide Acid); SAA-PEG-SAA (Bifunctional PEG carboxylic acid, Succinamide Acid); Azide-PEG-Azide (Bifunctional PEG azide, N3-PEG-N3); Alkyne-PEG-Alkyne (Bifunctional alkyne or acetylene PEG); Biotin-PEG-Biotin (Bifunctional biotin PEG linker); Silane-PEG-Silane (Bifunctional silane PEG); Hydrazide-PEG-Hydrazide (Bifunctional PEG Hydrazide); Tosylate-PEG-Tosylate (Bifunctional PEG Tosyl); and Chloride-PEG-Chloride (Bifunctional PEG Halide). Methods of conjugating PEG to a protein are standard in the art. For example, see Kolate et al, Journal of Controlled Release 2014; 192(28): 67-81, which is hereby incorporated by reference in its entirety. In certain embodiments, PEG is added to a peptide linker or alkyl chain linker. Different molecular weights of PEG may be used as a linker. The molecular weight of PEG may range from 200 g/mol to 10,000,000 g/mol. The linker may comprise PEG with a molecular weight of 200 g/mol, 300 g/mol, 400 g/mol, 500 g/mol, 1000 g/mol, 2000 g/mol, 3000 g/mol, 4000 g/mol, 5000 g/mol, 6000 g/mol, 7000 g/mol, 8000 g/mol, 9000 g/mol or 10,000 g/mol. In certain embodiments, the linker comprises PEG with a molecular weight of 200 g/mol, 500 g/mol, 2000 g/mol or 5000 g/mol.

In certain embodiments, the peptide is conjugated to the PEG linker via its N-terminus and the cypate is conjugated to the PEG linker via its C-terminus. Specifically, the C-terminus of the cypate is conjugated to the N-terminus of the Fmoc-PEG-COOH linker and then the C-terminus of the Fmoc-PEG-COOH linker is conjugated to the N-terminus of the peptide.

In other embodiments, a linker further comprises one or more spacers. Spacers are known in the art. Non-limiting examples of spacers include 2-aminoethoxy-2-ethoxy acetic acid (AEEA) linkers, AEEEA linkers, and AEA linkers.

Another aspect involves cross-linking the peptides of the disclosure to a linker, detectable label and/or therapeutic agent. Cross-linking involves joining two molecules by a covalent bond through a chemical reaction at suitable site(s) (e.g., primary amines, sulfhydryls) on the peptide and the linker, detectable label and/or therapeutic agent. In an embodiment, the peptide and the linker may be cross-linked together. In another embodiment, the peptide and the detectable label may be cross-linked together. In still another embodiment, the peptide and the therapeutic agent may be cross-linked together. The cross-linking agents may form a cleavable or non-cleavable linker between the peptide and the linker, detectable label and/or therapeutic agent. Cross-linking agents that form non-cleavable linkers between the peptide and the linker, detectable label and/or therapeutic agent may comprise a maleimido- or haloacetyl-based moiety. According to the present disclosure, such non-cleavable linkers are said to be derived from maleimido- or haloacetyl-based moiety. Cross-linking agents comprising a maleimido-based moiety include N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester [AMAS], succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). These cross-linking agents form non-cleavable linkers derived from maleimido-based moieties. Cross-linking agents comprising a haloacetyl-based moiety include N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido)propionate (SBAP). These cross-linking agents form non-cleavable linkers derived from haloacetyl-based moieties. Cross-linking agents that form non-cleavable linkers between the cytokine and the ligand may comprise N-succinimidyl 3-(2-pyridyldithio)propionate, 4-succinimidyl-oxycarbonyl-α-methyl-alpha-(2-pyridyldithio)-toluene (SMPT), N-succinimidyl-3-(2-pyridyldithio)-butyrate (SDPB), 2-iminothiolane, or acetylsuccinic anhydride.

(e) Pharmaceutical Composition

The peptide constructs of the present disclosure may further comprise a drug carrier to facilitate drug preparation and administration. Any suitable drug delivery vehicle or carrier may be used, including but not limited to a gene therapy vector (e.g., a viral vector or a plasmid), a microcapsule, for example a microsphere or a nanosphere (Manome et al., 1994; Hallahan, 2001a; Saltzman & Fung, 1997), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al., 1997 and U.S. Pat. Nos. 4,551,482, 5,714,166, 5,510,103, 5,490,840, and 5,855,900), and a polysome (U.S. Pat. No. 5,922,545).

Additionally, the peptide constructs may be formulated into pharmaceutical compositions and administered by a number of different means that may deliver a therapeutically effective dose. Such compositions may be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

Solid dosage forms for oral administration may include capsules, tablets, pills, powders, and granules. In such solid dosage forms, the peptide construct is ordinarily combined with one or more adjuvants appropriate to the indicated route of administration. If administered per os, the composition can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as can be provided in a dispersion of active composition of the invention in hydroxypropylmethyl cellulose. In the case of capsules, tablets, and pills, the dosage forms may also comprise buffering agents such as sodium citrate, or magnesium or calcium carbonate or bicarbonate. Tablets and pills may additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The amount of the peptide construct of the disclosure that may be combined with the carrier materials to produce a single dosage of the composition can and will vary depending upon the subject, the peptide, the formulation, and the particular mode of administration. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

In certain embodiments, a composition comprising a peptide construct of the disclosure is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating peptide constructs into delivery vehicles are known in the art.

In one alternative embodiment, a liposome delivery vehicle may be utilized. Liposomes, depending upon the embodiment, are suitable for delivery of the peptide construct of the invention in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the peptide construct of the disclosure may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palm itate), n-octadecanoate (stearate), n-eicosanoate (arachidate), n-docosanoate (behenate), n-tetracosanoate (lignocerate), cis-9-hexadecenoate (palm itoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the peptide construct of the disclosure (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a peptide construct of the disclosure may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The peptide construct of the disclosure may be encapsulated in a microemulsion by any method generally known in the art.

In yet another embodiment, a peptide construct of the disclosure may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate peptide constructs of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

### II. Methods

In an aspect, the disclosure provides a method to determine distribution of natriuretic peptide receptors, the method comprising administering a peptide composition of the disclosure to a subject and imaging the subject to detect the presence of a signal emitted from the peptide composition of the disclosure, wherein the signal being emitted is from binding of the peptide composition to one or more natriuretic peptide receptors. Specifically, a peptide composition of the disclosure comprises a NIR dye and the imaging is optical imaging. In a specific embodiment, the receptor is NPR—C and the peptide comprises CANP or a fragment thereof. Accordingly, a method of the disclosure may be used to image any pathological condition associated with expression of natriuretic peptide receptors. More specifically, a method of the disclosure may be used to image any pathological condition associated with expression of NPR-C when using a peptide composition of the disclosure comprising CANP or a fragment thereof.

In another aspect, the disclosure provides a method to determine distribution of an atherosclerotic plaque, the method comprising administering a peptide composition of the disclosure to a subject and imaging the subject to detect the presence of a signal emitted from the peptide composition of the disclosure, wherein the signal being emitted is from binding of the peptide composition to one or more natriuretic peptide receptors. An atherosclerotic plaque may be a stable plaque or an unstable plaque. An unstable plaque is a soft plaque and is more likely to rupture. An unstable plaque may also be referred to as a vulnerable plaque. The unstable plaque is an atheromatous plaque comprising a collection of white blood cells (primarily macrophages) and lipids (including cholesterol) in the wall of an artery. An unstable plaque is prone to produce sudden major problems such as a heart attack or stroke. A stable plaque is a calcified plaque and is less likely to rupture. In a specific embodiment, the atherosclerotic plaque is an unstable plaque.

In still another aspect, the disclosure provides a method to distinguish between an unstable and stable atherosclerotic plaque, the method comprising administering a peptide composition of the disclosure to a subject and imaging the subject to detect the presence of a signal emitted from the peptide composition of the disclosure, wherein detection of the signal indicates binding of the peptide composition to one or more natriuretic peptide receptors in an unstable atherosclerotic plaque. More specifically, the receptor is NPR—C and the peptide comprises CANP or a fragment thereof. NPR—C is present in the vascular smooth muscle cells (VSMC), endothelial cells and macrophages of complex atherosclerotic plaque. The presence of NPR-C indicates that the atherosclerotic plaque is unstable, may be about to rupture and could cause sudden myocardial infarction (heart attack) or stroke. Accordingly, when the peptide comprises CANP or a fragment thereof and a signal is detected, then the plaque is unstable and the subject is at risk of an atherosclerotic plaque rupture and myocardial infarction or stroke.

In still yet another aspect, the disclosure provides a method to determine risk of an impending atherosclerotic plaque rupture, the method comprising administering a peptide composition of the disclosure to a subject and imaging the subject to detect the presence of a signal emitted from the peptide composition of the disclosure, wherein detection of the signal indicates binding of the peptide composition to one or more natriuretic peptide receptors and risk of an impending atherosclerotic plaque rupture. The method may further comprise treatment of the subject if risk of an impending atherosclerotic plaque rupture is indicated. Notably, there is currently no way to identify a culprit lesion before it ruptures. Accordingly, compositions and methods of the present disclosure enable improved interventions or enable earlier interventions.

In a different aspect, the disclosure provides a method to image angiogenesis or atherosclerosis, the method comprising administering a peptide composition of the disclosure to a subject and imaging the subject to detect the presence of a signal emitted from the peptide composition of the disclosure, wherein the signal being emitted is from binding of the peptide composition to one or more natriuretic peptide receptors. In certain embodiments, angiogenesis is monitored during the course of anti-angiogenic treatment for cancer.

In another different aspect, the disclosure provides a method to monitor progression of atherosclerosis, the method comprising administering a peptide composition of the disclosure to a subject and imaging the subject to detect the presence of a signal emitted from the peptide composition of the disclosure. Then at a later time, again administering a peptide composition of the disclosure to a subject, imaging the subject to detect the presence of a signal emitted from the peptide composition of the disclosure, and determining the change in signal over time. For example, the subject may be imaged minutes, hours, days, weeks, months, or years following the initial imaging. Accordingly, the subject may be followed over time to determine when the atherosclerotic plaque becomes unstable or is at risk of rupturing. An increase in detectable signal suggests the atherosclerotic plaque is becoming unstable or is at risk of rupturing. In contrast, a decrease in detectable signal or no change in detectable signal suggests the atherosclerotic plaque is either becoming stable, not growing or is reducing in size. In certain embodiments, the subject is monitored during a stroke or heart attack.

Additionally, a method for monitoring progression of atherosclerosis in a subject may also be used to determine response to treatment. As used herein, subjects that respond to treatment are said to have benefited from treatment. Responses to treatment are measured in clinical practice using tests including, but not limited to, measuring blood pressure, blood tests to measure cholesterol, fat, sugar and protein, listening to the subject's arteries, checking the subject's pulses, electrocardiogram (EKG), chest X ray, ankle/brachial index, echocardiography, computed tomography scan, stress testing, and angiography. These tests are well known in the art and are intended to refer to specific parameters measured during clinical trials and in clinical practice by a skilled artisan. For example, a subject may be imaged prior to initiation of treatment. Then at a later time, a subject may be imaged to determine the response to treatment over time. For example, a subject may be imaged minutes, hours, days, weeks, months or years following initiation of treatment. Accordingly, a peptide composition of the disclosure may be used to follow a subject receiving treatment to determine if the subject is responding to treatment. If the signal detected increases, then the subject may not be responding to treatment. If the signal detected decreases or remains the same, then the subject may be responding to treatment. These steps may be repeated to determine the response to therapy over time.

The disclosure comprises, in part, imaging a subject. Non-limiting examples of modalities of imaging may include magnetic resonance imaging (MRI), ultrasound (US), computed tomography (CT), Positron Emission Tomography (PET), Single Photon Emission Computed Tomography (SPECT), optical imaging (01, bioluminescence and fluorescence), and fluorescence molecular tomography (FMT). Radioactive molecular probes are traditionally imaged with PET, SPECT or gamma (γ) cameras, by taking advantage of the capability of these imaging modalities to detect the high energetic γ rays. In contrast, 01 and FMT generally detects low energy lights (visible or near-infrared lights) emitted from bioluminescence or fluorescence probes. In a specific embodiment, the imaging modality is FMT.

The term “signal” as used herein, refers to a signal derived from a compound that can be detected and quantitated with regards to its frequency and/or amplitude. The signal can be generated from one or more peptide compositions of the present disclosure. In an embodiment, the signal may need to be the sum of each of the individual signals. In an embodiment, the signal can be generated from a summation, an integration, or other mathematical process, formula, or algorithm, where the signal is from one or more compounds. In an embodiment, the summation, the integration, or other mathematical process, formula, or algorithm can be used to generate the signal so that the signal can be distinguished from background noise and the like. It should be noted that signals other than the signal of interest can be processed and/or obtained in a similar manner as that of the signal of interest.

Using a method of the disclosure, microscopic plaques of atherosclerosis may be detected in a subject. Such plaques are generally not visible with current imaging techniques. Further the peptide compositions of the disclosure may be used to improve early diagnosis and interrogate the efficacy of therapeutics for the treatment of atherosclerosis.

In a different aspect, a method of the disclosure may be used to treat a pathological condition associated with expression of natriuretic peptide receptors, the method comprising administering a peptide composition of the disclosure to a subject, wherein the peptide comprises a therapeutic agent. More specifically, a method of the disclosure may be used to treat a pathological condition associated with expression of NPR-C when using a peptide composition of the disclosure comprising CANP or a fragment thereof. In a specific embodiment, the disclosure provides a method of treating atherosclerosis, the method comprising administering a peptide composition of the disclosure to a subject, wherein the peptide comprises CANP or a fragment thereof and a therapeutic agent. The term “treat”, “treating” or “treatment” as used herein refers to administering a peptide composition of the disclosure for prophylactic and/or therapeutic purposes. The term “prophylactic treatment” refers to treating a subject who is not yet experiencing symptoms, but who may have, or otherwise at a risk of having atherosclerosis. The term “therapeutic treatment” refers to administering treatment to a subject already suffering from atherosclerosis. The term “treat”, “treating” or “treatment” as used herein also refers to administering a compound of the disclosure in order to: (i) reduce or eliminate either atherosclerosis or one or more symptoms of atherosclerosis, or (ii) retard the progression of atherosclerosis or of one or more symptoms of atherosclerosis, or (iii) reduce the severity of atherosclerosis or of one or more symptoms of atherosclerosis, or (iv) suppress the clinical manifestation of atherosclerosis, or (v) suppress the manifestation of adverse symptoms of atherosclerosis.

In any of the foregoing embodiments, the subject may or may not be diagnosed with atherosclerosis. In certain embodiments, the subject may not be diagnosed with atherosclerosis but is suspected of having atherosclerosis based on symptoms. Non-limiting examples of symptoms of atherosclerosis that may lead to a diagnosis are known to those of skill in the art and may include angina, shortness of breath, arrhythmia, sleep problems, fatigue, lack of energy, sudden weakness, paralysis or numbness of the face, arms, legs, confusion, trouble speaking or understanding speech, trouble seeing in one or both eyes, problems breathing, dizziness, trouble walking, loss of balance or coordination, unexplained falls, loss of consciousness, sudden and severe headache, and loss of kidney function. In other embodiments, the subject may not be diagnosed with atherosclerosis but is at risk of having atherosclerosis. Non-limiting examples of risk factors for atherosclerosis are known to those of skill in the art and may include unhealthy blood cholesterol levels, high blood pressure, smoking, insulin resistance, diabetes, overweight or obesity, lack of physical activity, unhealthy diet, older age, family history of early heart disease, high levels of CRP, inflammation, high levels of triglycerides, sleep apnea, stress, and alcohol. In certain embodiments, a subject is at risk for rupturing an atherosclerotic plaque. In another embodiment, a subject is in the process of rupturing an atherosclerotic plaque In other embodiments, the subject has no symptoms and/or no risk factors for atherosclerosis. Methods of diagnosing atherosclerosis are known to those of skill in the art and may include measuring blood pressure, blood tests to measure cholesterol, fat, sugar and protein, listening to the subject's arteries, checking the subject's pulses, electrocardiogram (EKG), chest X ray, ankle/brachial index, echocardiography, computed tomography scan, stress testing, and angiography.

Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In preferred embodiments, the animal is a laboratory animal. Non-limiting examples of a laboratory animal may include rodents, canines, felines, and non-human primates. In certain embodiments, the animal is a rodent. In a preferred embodiment, the subject is human.

In certain aspects, a pharmacologically effective amount of a compound of the disclosure may be administered to a subject. Administration is performed using standard effective techniques, including peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the compounds useful in this discovery, making them more lipophilic, for example, by encapsulating them in liposomes or by blocking polar groups.

Effective peripheral systemic delivery by intravenous or intraperitoneal or subcutaneous injection is a preferred method of administration to a living patient. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hem isuccinate, phosphatidyl glycerols and the like.

For diagnostic applications, a detectable amount of a compound of the disclosure is administered to a subject. A “detectable amount”, as used herein to refer to a diagnostic composition, refers to a dose of such a compound that the presence of the compound can be determined in vivo or in vitro. A detectable amount will vary according to a variety of factors, including but not limited to chemical features of the compound, labeling methods, the method of imaging and parameters related thereto, metabolism of the compound in the subject, the stability of the compound (e.g. the half-life of a detectable label), the time elapsed following administration of the compound prior to imaging, the route of drug administration, the physical condition and prior medical history of the subject, and the size and longevity of the atherosclerotic plaque or suspected atherosclerotic plaque. Thus, a detectable amount can vary and can be tailored to a particular application. After study of the present disclosure, and in particular the Examples, it is within the skill of one in the art to determine such a detectable amount. A detectable amount may be visible from about 1 to about 120 hours or more. For example, a detectable amount may be visible from about 1 to about 110 hours, or from about 1 to about 100 hours. Accordingly, a detectable amount may be visible at about 1, about 2, about 3, about 4, about 5, about 6, about 7, about 8, about 9, about 10, about 11, about 12, about 16, about 24, about 36, about 48, about 60, about 72, about 84, about 96, about 108, or about 120 hours.

For therapeutic applications, a therapeutically effective amount of a compound of the disclosure is administered to a subject. A “therapeutically effective amount” is an amount of the therapeutic composition sufficient to produce a measurable biological response (e.g., reduction or no change in atherosclerotic plaque size, reduction in symptoms associated with atherosclerosis). Actual dosage levels of active ingredients in a therapeutic composition of the disclosure can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, atherosclerotic plaque size and longevity, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be daily or once, twice, three times or more per week or per month, as needed as to effectively treat the symptoms. The timing of administration of the treatment relative to the disease itself and duration of treatment will be determined by the circumstances surrounding the case. Treatment could begin immediately. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Although the foregoing methods appear the most convenient and most appropriate and effective for administration of peptide constructs, by suitable adaptation, other effective techniques for administration, such as intraventricular administration, transdermal administration and oral administration may be employed provided proper formulation is utilized herein.

In addition, it may be desirable to employ controlled release formulations using biodegradable films and matrices, or osmotic mini-pumps, or delivery systems based on dextran beads, alginate, or collagen.

Typical dosage levels can be determined and optimized using standard clinical techniques and will be dependent on the mode of administration.

In certain aspects, the method of the disclosure may further comprise additional diagnostic tests. Methods of diagnosing atherosclerosis are known to those of skill in the art and may include measuring blood pressure, blood tests to measure cholesterol, fat, sugar and protein, listening to the subject's arteries, checking the subject's pulses, electrocardiogram (EKG), chest X ray, ankle/brachial index, echocardiography, computed tomography scan, stress testing, and angiography.

In certain aspects, the methods of the invention may further comprise administering therapy standard for the treatment of atherosclerosis. Suitable therapy for atherosclerosis is known in the art, and will depend upon the type and stage of atherosclerosis. Non-limiting examples of therapy for atherosclerosis include heart-healthy lifestyle changes such as heart-healthy eating, aiming for a healthy weight, managing stress, physical activity and quitting smoking, statins, percutaneous coronary intervention (PCI), coronary artery bypass grafting (CABG), bypass grafting, and carotid endarterectomy.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Noninvasive Imaging of Focal Atherosclerotic Lesions Using Fluorescence Molecular Tomography

Carotid artery atherosclerosis is classified as an important cause of stroke. Unstable plaque is characterized by an eccentric neo-intimal lesion with a lipid core covered by a thinning cap of smooth muscle cells, active angiogenesis, increased matrix metalloproteinase activity, and translocation of monocyte/macrophages that transform into foam cells. Timely noninvasive imaging that could signal prerupture plaque progression will reduce the morbidity and mortality by allowing early intervention.¹ Although positron emission tomography (PET) and magnetic resonance imaging (MRI) are routinely used for metabolic and morphologic imaging, these modalities are not suited for frequent monitoring or even screening of at-risk patients because of ionizing radiation (PET) and expense (PET, MRI). Transcutaneous Doppler and intravascular ultrasound are insensitive to the subtle molecular changes of critical importance. Fluorescence molecular tomography (FMT) is an emerging optical imaging technology that allows tomographic and quantitative visualization of molecular events in vivo.² In this study, a custom built, fiber-based, portable, video-rate FMT system was used for proof-of-principle studies to detect C-type natriuretic peptide receptors (NPR-C) on focal atherosclerotic lesions in the superficial rabbit femoral arteries. The FMT system consisted of a flexible imaging pad (3 cm×3 cm), containing 12 sources 785 nm 20 kHz, and 830 nm 17-kHz laser diodes as excitation source and reference, respectively.³ The detectors allowed for dynamic concurrent acquisition of frequency encoded fluorescence emission (830 nm; 20 kHz) and transmission reference (830 nm; 17 kHz) signals for the fast generation of ratio-metric data for tomographic reconstruction of the tissue volume. The system could report varying concentrations (1 nM to 1 M) of indocyanine green at various depths up to 13.5 mm with a depth-dependent spatial resolution on the order of 12 mm.³

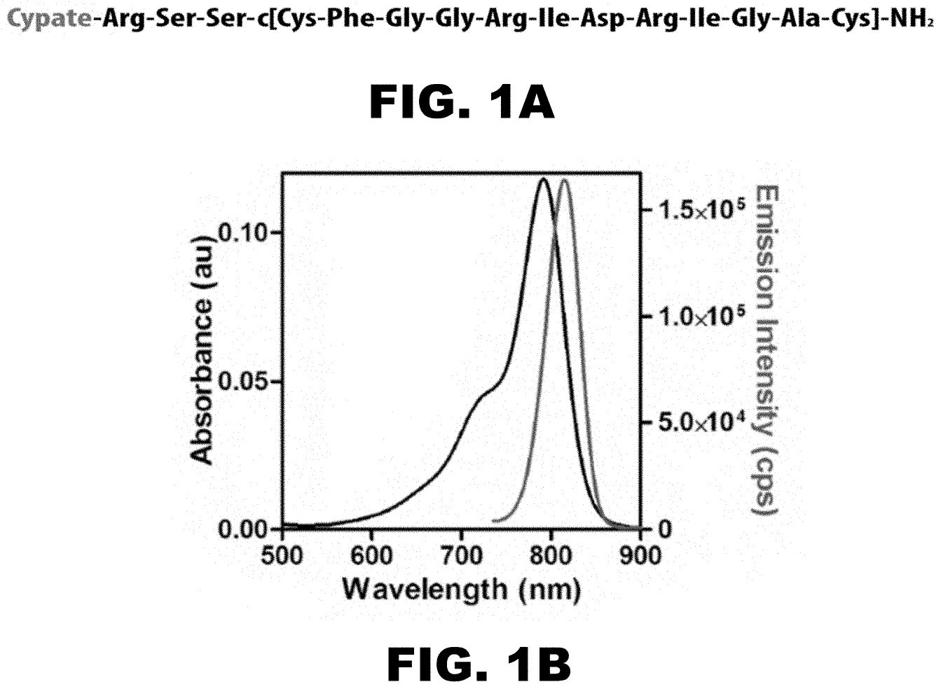

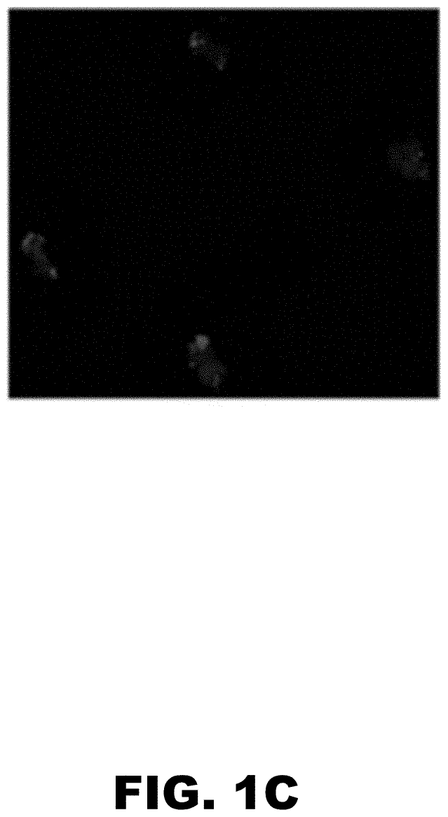

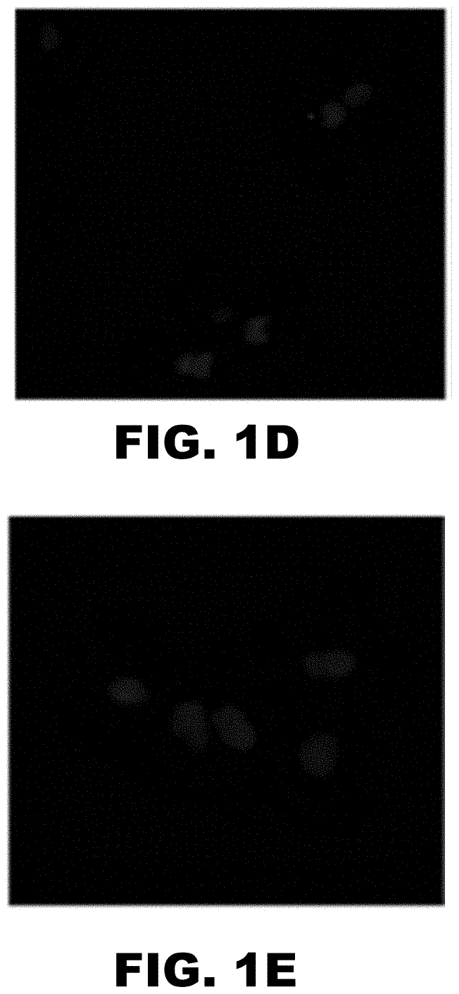

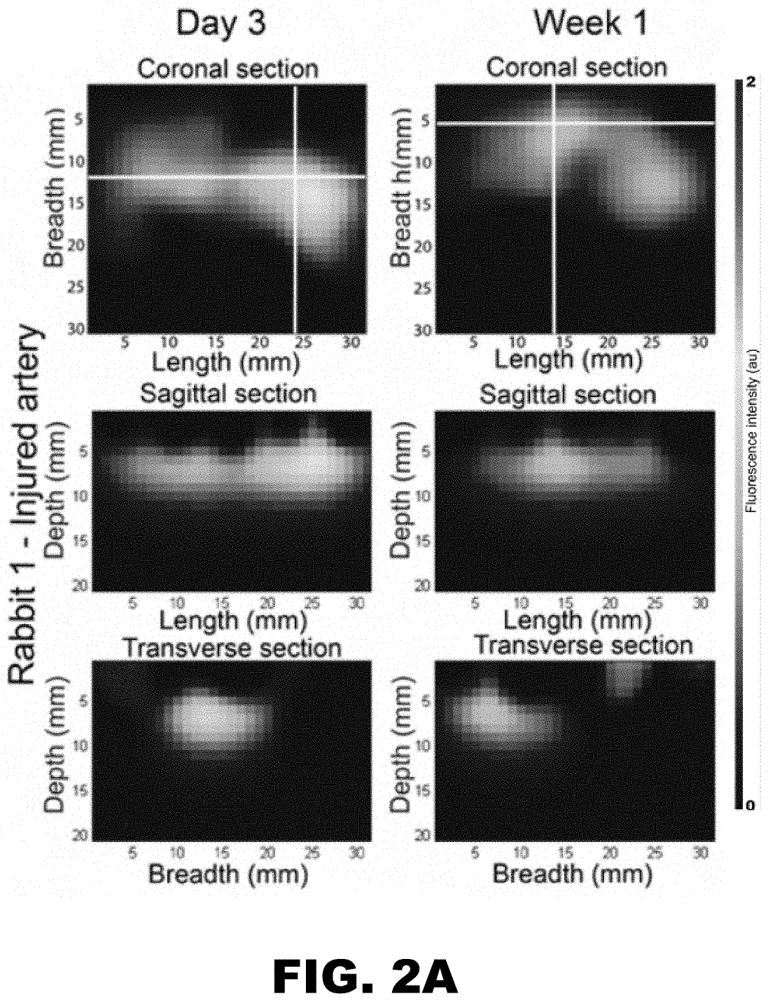

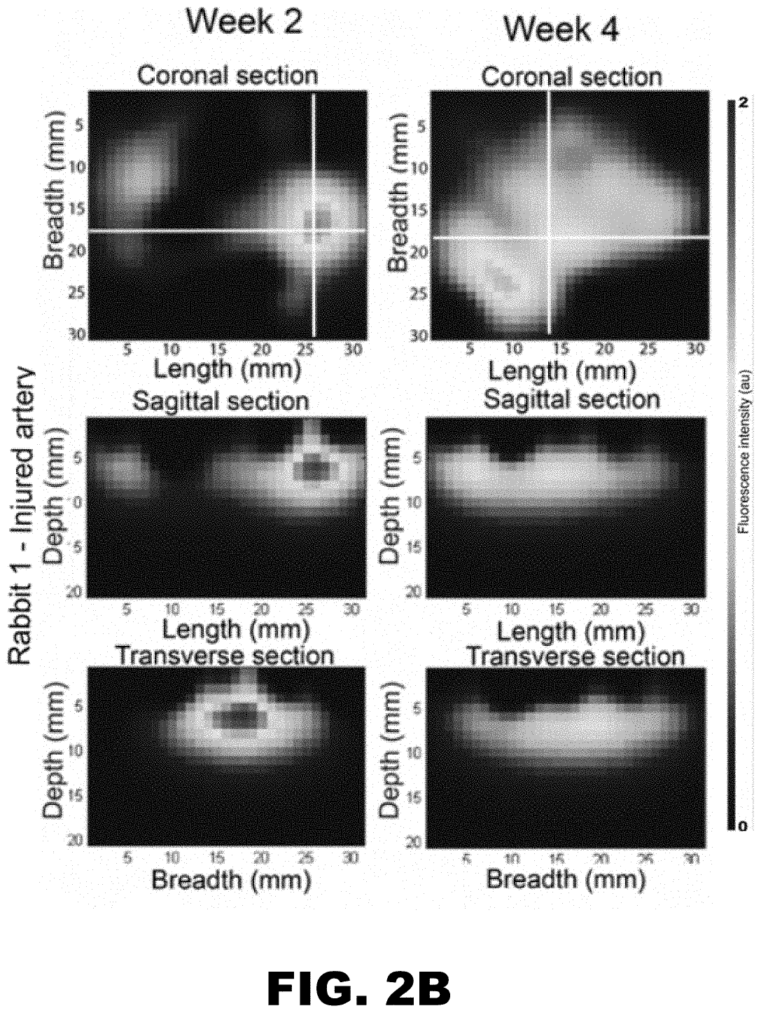

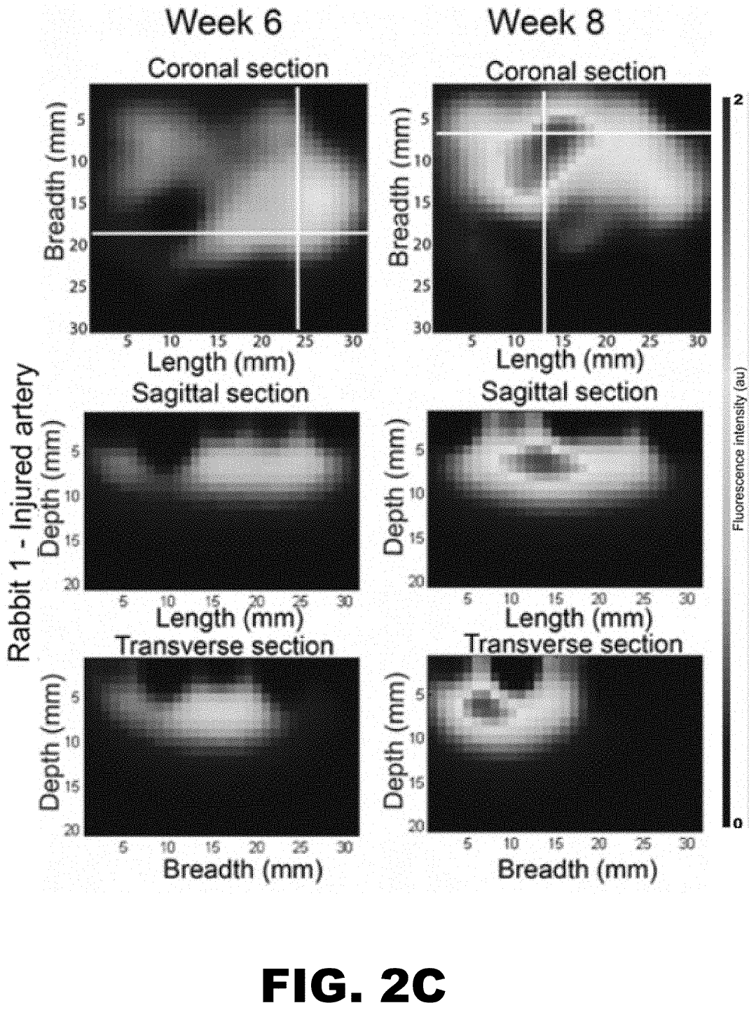

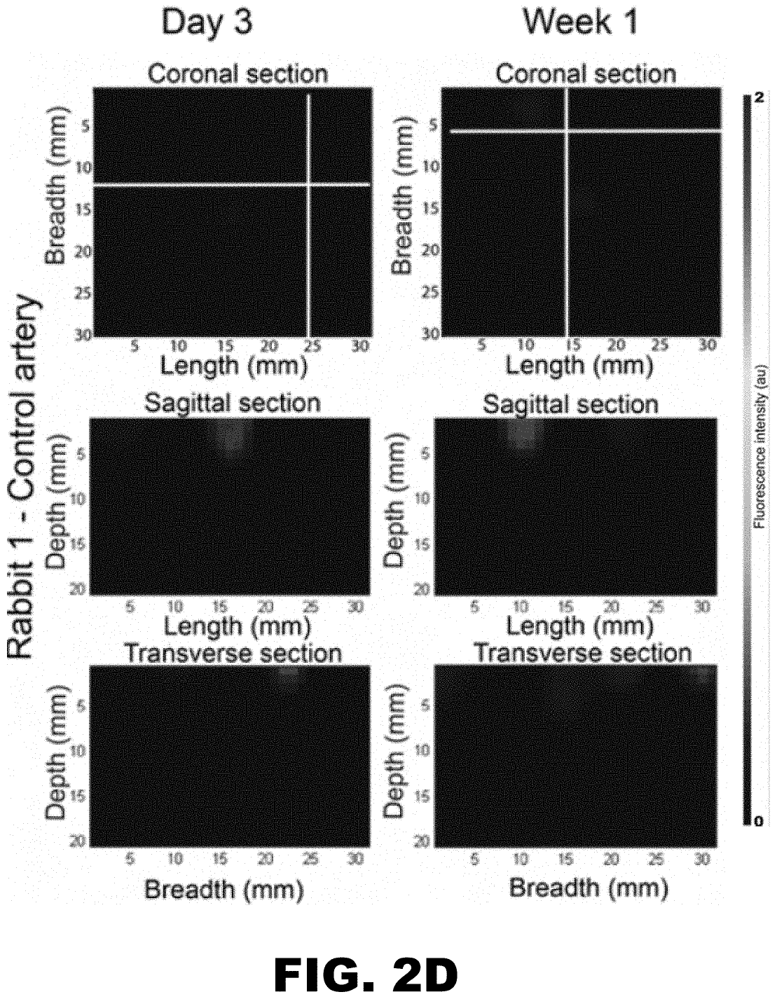

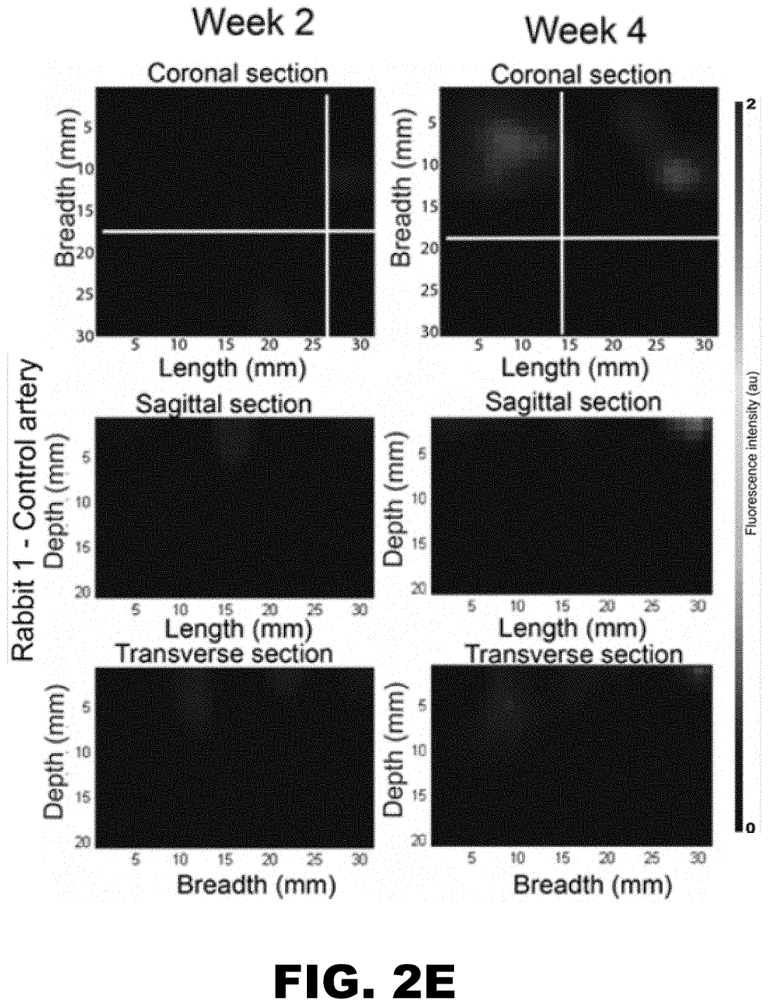

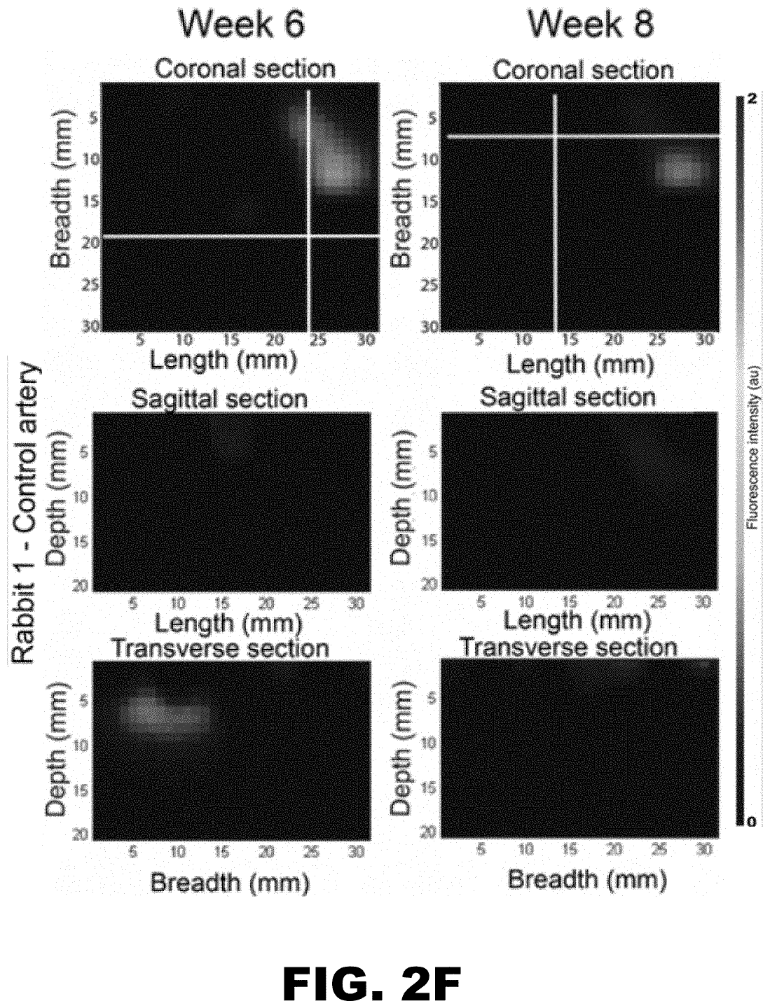

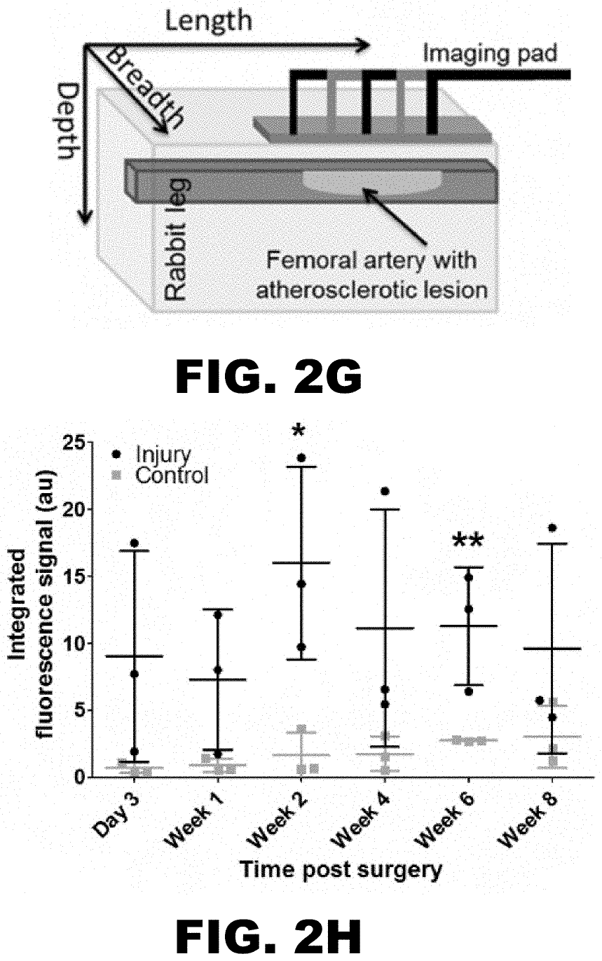

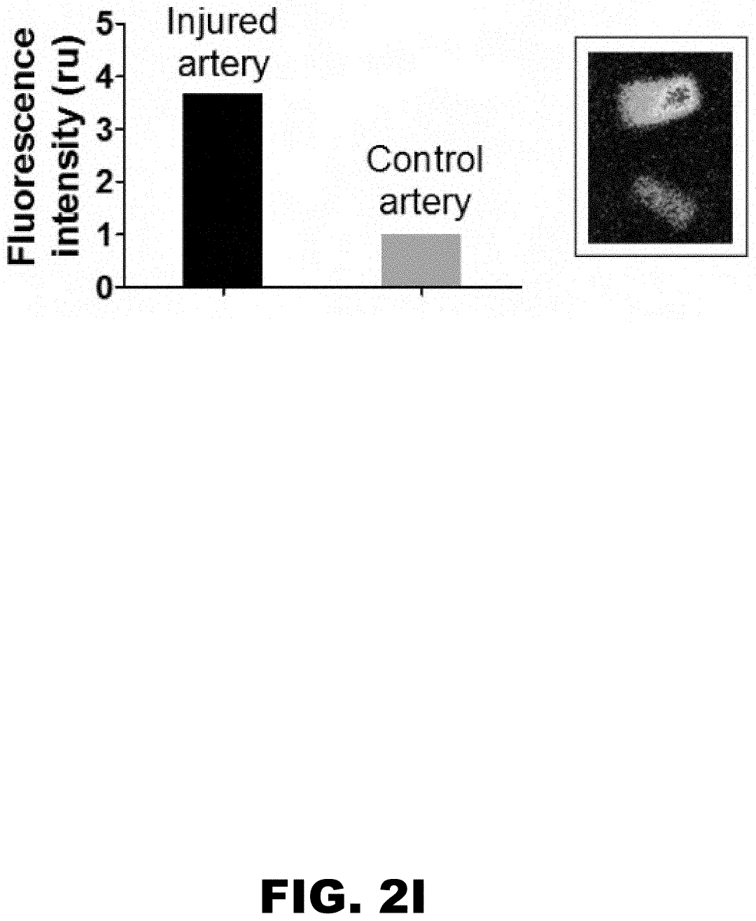

A model of focal atherosclerotic-like plaques in the femoral arteries (located 1 to 1.5 cm from skin surface) of New Zealand white rabbits was used. All animal studies performed were approved by the Washington University Animal Studies Committee. Endothelial denudation of surgically exposed right femoral artery was induced by air desiccation of the luminal surface as described previously.⁴ The uninjured left femoral artery served as the internal control. The animals were maintained on a cholesterol-enriched diet (>200 mg dL in blood), and over time the air desiccation led to a focal lesion. In this animal model, anatomical coregistration was not used because the location of the lesion was apparent due to the surgical markings and identification by the surgeon at each imaging session. In a clinical setting, coregistration of the FMT probe with the ultra sound (US) probe can be utilized. The progression of the receptor, NPR-C, was monitored, which has been shown to undergo changes during atherosclerotic plaque progression and was recently evaluated as a PET imaging marker.⁵ NPR-C is a cell surface protein found on endothelial, vascular smooth muscle, and macrophage cells. Natriuretic peptides (NPs) play an important role in regulating cardiovascular homeostasis. NPR-C (clearance receptor) removes NPs from circulation by receptor mediated endocytosis.⁶ A bioconjugate LS668 (Cypate-RSSc[CFGGRIDRIGAC]) (SEQ ID NO:3), consisting of a near infrared (NIR) fluorescent dye cypate conjugated to a targeting peptide, C-type atrial natriuretic peptide, specific for NPR-C was evaluated (FIG. 1A). NIR fluorescent (700 to 900 nm) imaging agents are desirable for in vivo imaging due to enhanced depth penetration of NIR light and low tissue autofluorescence.⁷ Cell studies demonstrated that LS668 (1 M, 30-min incubation at 37° C., 5% CO₂) was selectively internalized by stably transfected 293T-NPR-C cells (FIG. 1B).⁸ Internalization was blocked in the presence of excess (100 M) C-ANF peptide (FIG. 1C), and additionally, LS668 did not internalize into the control 293T-NPR-A cells (FIG. 1D), supporting receptor mediated endocytosis of LS668. For in vivo imaging, 24-h post-injection of LS668 was selected as the optimal time point as clearance of LS668 from blood was achieved at 24 h. Both injured and control femoral arteries of three animals were imaged at day 3 and weeks 1, 2, 4, 6, and 8 following the surgery. For each time point, LS668 (0.1 mg kg intravenous) was injected and 24 h later FMT scans were performed (5 min each) in triplicates for each artery. FMT reconstruction was performed as reported earlier to obtain three-dimensional (3-D) data from the tissue containing the lesion.³ FMT scans of the respective arteries before surgery were used as blank scans for image reconstruction. In the reconstructed data, localized fluorescence signal indicating accumulation of LS668 was observed at a depth of 4 to 16 mm and over 15 mm of length consistent with the location of the focal lesion. Coronal section images of the 3-D volumes are shown at a depth of 7 mm for one representative animal (FIG. 2A-FIG. 2F). The corresponding sagittal and transverse sections show the spread of the lesion along the length and breadth (FIG. 2A-FIG. 2G). Near background signal from the tissue surrounding the localized fluorescent region indicated negligible nonspecific uptake by surrounding tissue. Contralateral noninjured femoral arteries showed minimal signal indicating negligible background uptake of LS668 in the control artery. Integrated fluorescence signal (directly related to the quantity of LS668 in tissue) was calculated from each tissue volume (thresholded at above 20% of respective maximum signal) (FIG. 2H). Unpaired t test (two tailed) showed statistically significant difference between control and injured arteries at week 2 (P=0.0283) and week 6 (P=0.0282) (FIG. 2H). The changes in the signal over time most likely result from a transient increase in macrophages after injury usually at week 2, followed by a decrease resulting from the increased amount of the matrix and decrease in cellularity. Inflammation resumes in the following days or weeks due to the diet-induced macrophage-enriched unstable lesions. The variance within the cohorts highlights the differences in the pace of lesion formation in individual animals, probably as a function of their cholesterol levels. Ex vivo tissue biodistribution even after 24-h post-injection at week 8 showed 3.7-fold (n=2) higher localization of LS668 into the injured femoral artery as compared to the control femoral artery (FIG. 2I).

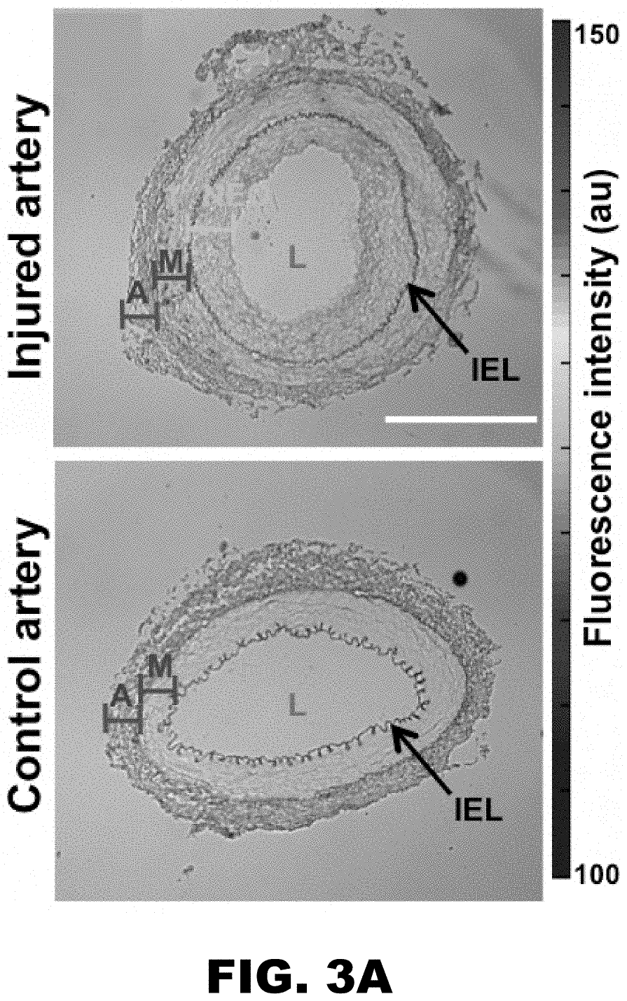

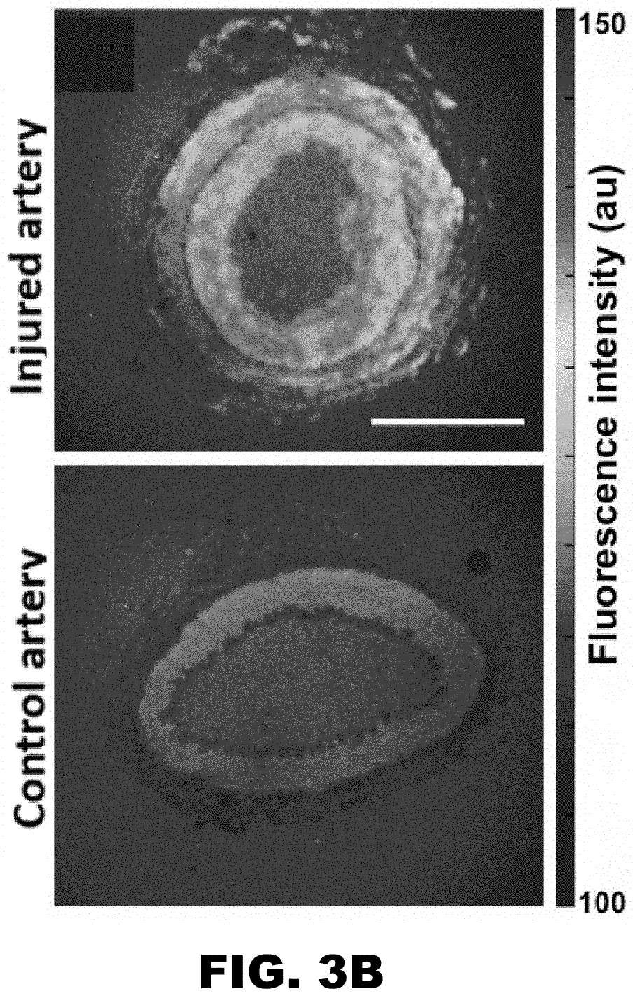

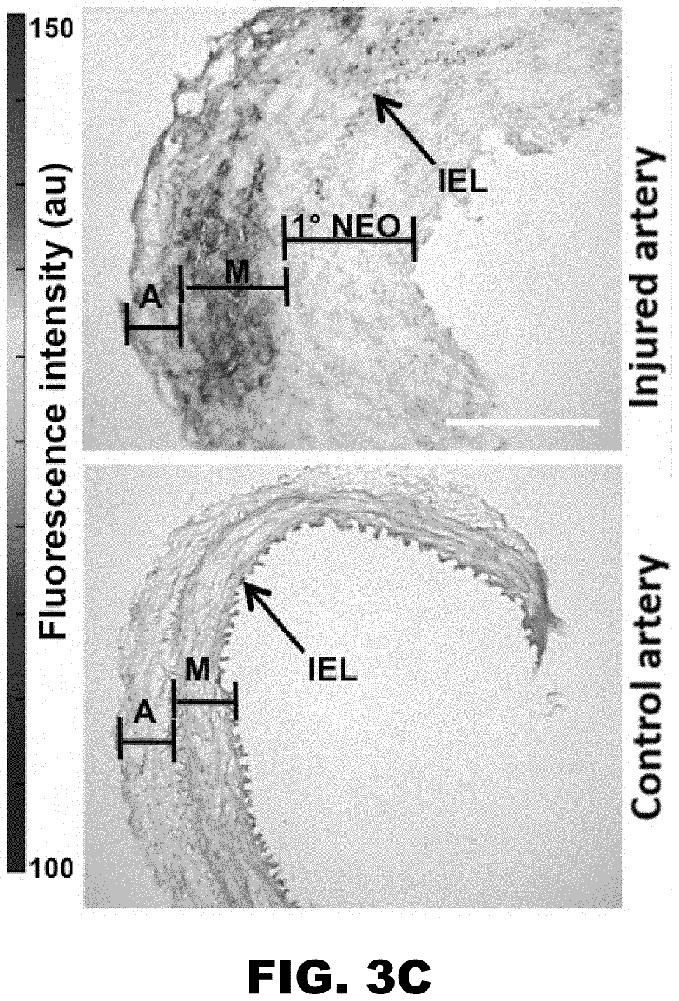

Ex vivo histological validation studies were performed on the arteries at week 8 following the final imaging session. Prior to collecting the arterial segments, the vascular system was flushed with saline and then perfusion fixed with 4% paraformaldehyde and paraffin embedded. Tissue sections were deparaffinized for further studies. The bright-field images of the injured artery showed a thick concentric layer of primary neointima (1° NEO) within the internal elastic lamina (IEL) (FIG. 3A). The control artery showed an intact adventitia (A), media (M), and IEL. Ex vivo staining of the injured artery section with LS668 (10 M; 30 min; 37° C.) followed by fluorescence microscopy showed an increased signal in the layers between A and the lumen (L), which is most likely due to the presence of infiltrating NPR-C expressing macrophages migrating from the adventitia through the media (smooth muscle cells) to accumulate in the base of the NEO closest to the media (FIG. 3B). The control section had a uniform fluorescence signal akin to nonspecific background. Histology sections were also stained for macrophages with mouse monoclonal antibody to rabbit macrophages (clone RAM11). Positive signal was visualized using alkaline phosphatase-conjugated secondary antibody and blue substrate, and nuclear fast red counterstain. In the injured artery, IHC showed a thickened adventitial layer and neointima with a dense accumulation of infiltrating macrophages primarily in the adventitial and medial layers of the injured femoral artery, and also some in the neointima (FIG. 3C). The control artery showed a negligible staining for macrophages. Serial sections were also stained for alpha-smooth muscle actin (SMA). The injured artery demonstrated medial hypertrophy and the staining for alpha-SMA was also markedly higher as compared to the control vessel.

In summary, noninvasive FMT study of atherosclerotic lesions was conveniently performed weekly/biweekly; a feature that is not practical for performing similar PET studies. Sequential imaging with FMT over several weeks showed quantifiable relative changes in the fluorescence intensity that provided insights into receptor concentration as the lesion progressed. This pilot study demonstrates a unique application of an FMT system for serial imaging of focal atherosclerotic plaques in the shallow femoral arteries in a rabbit model of atherosclerosis using a targeted NIR-fluorescent molecular imaging probe. Future studies will incorporate additional NIR-fluorescent imaging agents to serially evaluate the progression of high-risk plaques.

### REFERENCES FOR THE EXAMPLES

1. T. Quillard and P. Libby, “Molecular imaging of atherosclerosis for improving diagnostic and therapeutic development,” Circ. Res. 111(2), 231-244 (2012).
2. R. Weissleder and V. Ntziachristos, “Shedding light onto live molecular targets,” Nat. Med. 9(1), 123-128 (2003).
3. M. Solomon et al., “Video-rate fluorescence diffuse optical tomography for in vivo sentinel lymph node imaging,” Biomed. Opt. Express 2(12), 3267-3277 (2011).
4. D. Recchia et al., “The biologic behavior of balloon hyperinflation-induced arterial lesions in hypercholesterolemic pigs depends on the presence of foam cells,” Arterioscler. Thromb. Vasc. Biol. 15(7), 924-929 (1995).
5. Y. Liu et al., “Molecular imaging of atherosclerotic plaque with 64Cu-labeled natriuretic peptide and PET,” J. Nuclear Med. 51(1), 85-91 (2010).
6. T. Maack, “The broad homeostatic role of natriuretic peptides,” Arq. Bras. Endocrinol. Metabol. 50(2), 198-207 (2006).
7. J. V. Frangioni, “In vivo near-infrared fluorescence imaging,” Curr. Opin. Chem. Biol. 7(5), 626-634 (2003).
8. D. M. Dickey, D. R. Flora, and L. R. Potter, “Antibody tracking demonstrates cell type-specific and ligand-independent internalization of guanylyl cyclase a and natriuretic peptide receptor C,” Mol. Pharmacol. 80(1), 155-162 (2011).

What is claimed is:

1. A composition, the composition comprising a peptide conjugated to an optical imaging agent, wherein the peptide is capable of binding C-type natriuretic peptide receptors (NPR-C) and comprises the sequence Arg-Ile-Asp-Arg-Ile (SEQ ID NO:1) and wherein the optical imaging agent is a near infrared (NIR) dye.

2. The composition of claim 1, wherein the peptide is about 5 to about 25 amino acids.

3. The composition of claim 1, wherein the peptide comprises the sequence Cys-Phe-Gly-Gly-Arg-Ile-Asp-Arg-Ile-Gly-Ala-Cys (SEQ ID NO:2).

4. The composition of claim 3, wherein the first and second Cys form a disulfide linkage.

5. The composition of claim 3, wherein the carboxy terminus is aminated.

6. The composition of claim 1, wherein the peptide comprises the sequence Arg-Ser-Ser-c[Cys-Phe-Gly-Gly-Arg-Ile-Asp-Arg-Ile-Gly-Ala-Cys]-NH2 (SEQ ID NO:3).

7. The composition of claim 1, wherein the NIR dye is a cypate.

8. The composition of claim 1, wherein the NIR dye is conjugated to the N-terminus of the peptide.

9. The composition of claim 1, wherein the composition consists of Cypate-Arg-Ser-Ser-c[Cys-Phe-Gly-Gly-Arg-Ile-Asp-Arg-Ile-Gly-Ala-Cys]-NH2 (SEQ ID NO:3).

10. The composition of claim 1, wherein the peptide is conjugated to the optical imaging agent via a polyethylene glycol linker with a molecular weight of 200 g/mol, 500 g/mol, 2000 g/mol or 5000 g/mol.

11. The composition of claim 1, wherein the composition is a transdermal composition.

12. A method to determine distribution of C-type natriuretic peptide receptors (NPR-C) in a subject, the method comprising:
a) administering to the subject a peptide conjugated to an optical imaging agent, wherein the peptide is capable of binding C-type natriuretic peptide receptors (NPR-C) and comprises the sequence Arg-Ile-Asp-Arg-Ile (SEQ ID NO:1) and wherein the optical imaging agent is a NIR dye; and
b) imaging the subject to detect the presence of a signal emitted from the peptide, wherein the signal being emitted is from binding of the peptide to one or more C-type natriuretic peptide receptors.

13. The method of claim 12, wherein the peptide comprises the sequence Arg-Ser-Ser-c[Cys-Phe-Gly-Gly-Arg-Ile-Asp-Arg-Ile-Gly-Ala-Cys]-NH2 (SEQ ID NO:3).

14. The method of claim 12, wherein the imaging is optical imaging.

15. The method of claim 12, wherein the imaging is fluorescence molecular tomography (FMT).

16. The method of claim 12, wherein the method determines distribution of an atherosclerotic plaque or angiogenesis.

17. The method of claim 12, wherein the method distinguishes between an unstable and stable atherosclerotic plaque, wherein detection of the signal indicates an unstable atherosclerotic plaque.

18. A method to treat a pathological condition associated with expression of C-type natriuretic peptide receptors (NPR-C) in a subject, the method comprising administering to the subject a peptide conjugated to a therapeutic agent, wherein the peptide is capable of binding C-type natriuretic peptide receptors (NPR-C) and comprises the sequence Arg-Ile-Asp-Arg-Ile (SEQ ID NO:1).

19. The method of claim 18, wherein the peptide comprises the sequence Arg-Ser-Ser-c[Cys-Phe-Gly-Gly-Arg-Ile-Asp-Arg-Ile-Gly-Ala-Cys]-NH2 (SEQ ID NO:3).

20. The method of claim 18, wherein the pathological condition is atherosclerosis.


SEQUENCE LISTING (3 sequences)

SEQ ID NO: 1 - protein, 5 aa, Artificial Sequence
  SYNTHESIZED
RIDRI

SEQ ID NO: 2 - protein, 12 aa, Artificial Sequence
  SYNTHESIZED
CFGGRIDRIGAC

SEQ ID NO: 3 - protein, 15 aa, Artificial Sequence
  SYNTHESIZED
RSSCFGGRIDRIGAC